(19) United States
(12) Patent Application Publication
(10) Pub. No.: US 2008/0142011 A1
(43) Pub. Date: Jun. 19, 2008

(54) System and method of a positive airway pressure device gathering data as to apnea type

(75) Inventors: Alonzo C. Aylsworth, Wildwood, MO (US); Charles R. Aylsworth, Weldon Spring, MO (US)

Correspondence Address: CONLEY ROSE, P.C.;David A. Rose, P. O. BOX 3267, HOUSTON, TX, 77253-3267, US

(73) Assignee: ACOBA, L.L.C., Chesterfield, MO (US)

(21) Appl. No.: 11/958,474

(22) Filed: Dec. 18, 2007

Related U.S. Application Data
(60) Provisional application No. 60/870,665, filed on Dec. 19, 2006.

(51) Int. Cl. A61M 16/00

(52) U.S. Cl. 128/204.23

(57) ABSTRACT

System and method of a positive airway pressure device gathering data as to apnea type. At least some of the illustrative embodiments are systems comprising a processor, and a blower mechanically coupled to a motor (the blower configured to fluidly couple to a breathing orifice of a patient, and the blower provides substantially all gas inhaled by the breathing orifice). The processor is configured to monitor for an apnea event of the patient, and when an apnea event is detected the processor is configured to gather data indicative of whether the apnea is central apnea or obstructive apnea.

### CROSS-REFERENCE TO RELATED APPLICATIONS

This application claims the benefit of provisional application Ser. No. 60/870,665, filed Dec. 19, 2007, titled “System and method of a positive airway pressure device based central versus apnea determination”, and which provisional application is incorporated by reference herein as if reproduced in full below.

### BACKGROUND

Sleep apnea is defined in the field of respiratory therapy as a cessation of breathing during sleep lasting ten seconds or more. Sleep apnea may be characterized as either “central apnea” or “obstructive apnea.” Obstructive apnea is so named because the cessation of breathing is caused by an obstruction in the respiratory tract. For example, portions of the soft palate may collapse, blocking the airway. In the case of obstructive apnea, the patient may attempt to inhale (i.e. has breathing effort), but the blockage prevents inhalation. Central apnea occurs when a sleeping person's central nervous system fails to instruct the diaphragm to retract to draw air into the lungs.

One treatment for obstructive sleep apnea is use of a positive airway pressure device during sleep. A positive airway pressure device applies a positive airway pressure to the patient's nose and/or mouth during respiration. The positive airway pressure opens an obstructed airway, pneumatically splints the airway open to prevent collapse and thus obstruction, or both. In the case of continuous positive airway pressure devices (CPAP), the applied pressure is the same both during the inhalation and exhalation. In other cases the pressure applied during exhalation is lower than the pressure applied during inhalation, possibly to reduce the backpressure against which the patient exhales.

If the pressure applied by a positive airway pressure device is too high, the pressure may trigger a central apnea. While over-pressure triggered central apnea is possible with any type of positive airway pressure device (even those whose primary control parameter is airflow and only secondarily pressure), over-pressure triggered central apnea is particularly prevalent in positive airway pressure devices that automatically adjust applied pressure throughout the night. Raising applied pressure in response to an apnea may exacerbate the problem if the apnea is central, but raising the pressure is the proper response when the apnea is obstructive. Conversely, lowering applied pressure in response to an apnea is the proper response if the apnea is central, but lowering the applied pressure exacerbates the problem if the apnea is obstructive. Thus, the related-art approach is simply to set the applied pressure at a predetermined value (e.g., somewhere in the range of 10 to 12 inches of water), and wait for the apnea to cease.

### BRIEF DESCRIPTION OF THE DRAWINGS

For a detailed description of exemplary embodiments of the invention, reference will now be made to the accompanying drawings in which:

FIG. 1 shows system in accordance with some embodiments;

FIG. 2 shows a graph of flow rate as a function of time for normal breathing, and for an apnea event;

FIG. 3 shows a graph of flow rate as a function of time;

FIG. 4 shows a graph of pressure as function of time;

FIG. 5 shows a method in accordance with at least some embodiments.

### NOTATION AND NOMENCLATURE

Certain terms are used throughout the following description and claims to refer to particular system components. As one skilled in the art will appreciate, medical device companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . ”

Also, the term “couple” or “couples” is intended to mean either an indirect or direct connection. Thus, if a first device couples to a second device, that connection may be through a direct connection or through an indirect connection via other devices and connections.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

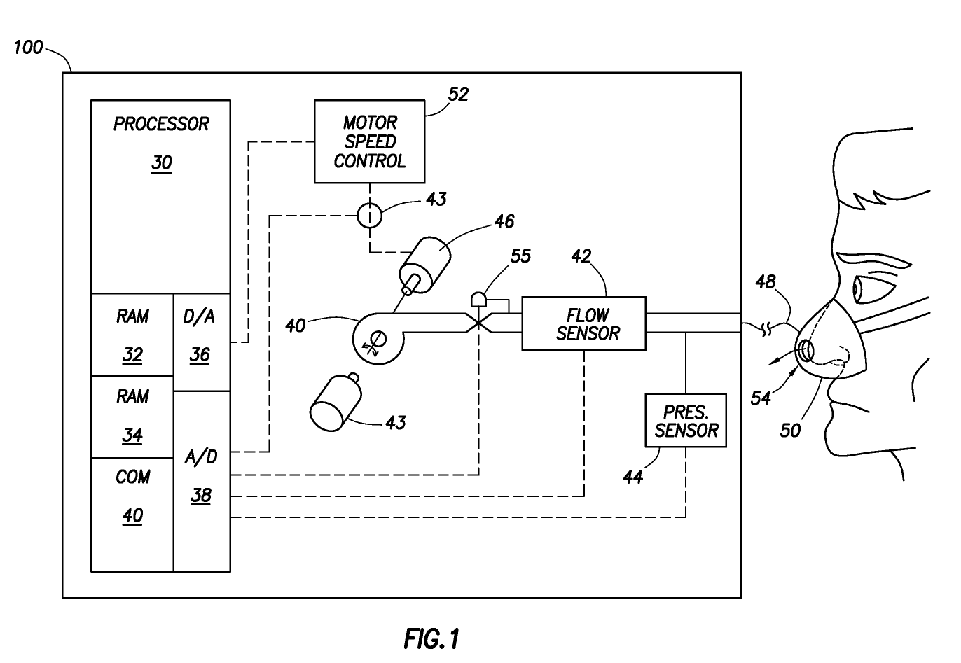

FIG. 1 illustrates a positive airway pressure device 100 in accordance with at least some embodiments. The positive airway pressure device 100 comprises both electrical components and mechanical components. In order to differentiate between electrical connections and mechanical connections, FIG. 1 illustrates electrical connections between components with dashed lines, and fluid connections (e.g., tubing connections between devices) with solid lines. The positive airway pressure device 100 in accordance with at least some embodiments comprises a processor 30. The processor 30 may be a microcontroller, and therefore the microcontroller may be integral with read-only memory (ROM) 32, random access memory (RAM) 34, a digital-to-analog converter (D/A) 36, and an analog-to-digital converter (A/D) 38. The processor 30 may further comprise a communications logic 40, which allows the positive airway pressure device 100 to communicate with external devices (e.g., to communicate results of testing performed during apnea events). In alternative embodiments the processor 30 may be implemented as a standalone central processing unit in combination with individual RAM, ROM, communications, D/A and A/D devices.

The ROM 32 stores instructions executable by the processor 30. In particular, the ROM 32 comprises a software program that implements the various embodiments of gathering data regarding apnea events, and in some cases testing to determine whether the apnea is central apnea or obstructive apnea. The RAM 34 may be the working memory for the processor 30, where data is temporarily stored and from which instructions are executed. Processor 30 may couple to other devices within the system by way of the A/D converter 38 and the D/A converter 36.

The positive airway pressure device in accordance with the various embodiments also comprises a fan or blower 40. Blower 40 is any suitable device, such as a vane-type blower, coupled to an electric motor 46. In alternative embodiments, a source of therapeutic gas (e.g., oxygen) may be used in addition to or in combination with the blower 40. Therapeutic gas pressure and flow created by the blower 40 flows through an optional flow sensor 42 and an optional pressure sensor 44. The therapeutic gas pressure and flow then couple to a breathing orifice of the patient, where all or substantially all the gas inhaled by the patient is supplied from the blower 40 or other gas source. In most situations, all the therapeutic gas (e.g., air) inhaled by the patient is provided by the blower 40; however, in some situations additional therapeutic gas (e.g., oxygen) is added to the therapeutic gas stream between the positive airway pressure device 100 and the patient (i.e., in the hose 48 coupling the device 100 to the patient), but for purposes of this disclosure and the claims positive airway pressure device 100 is still considered to provide substantially all the gas inhaled by the patient.

In the illustrative case of FIG. 1, the blower sealingly couples to the nose of the patient through tube and mask 50, but in alternative embodiments blower sealingly couples to the patient's mouth, or both the patient's nose and mouth. In order for carbon dioxide exhaled by the patient to escape the mask 50 during exhalation, a vent 54 is provided. For purposes of this disclosure and the claims, the fluid connection between the blower and the patient is still considered sealingly coupled in spite of the vent 54 in the mask 50 and any leaks at the interface of the patient and the mask 50.

In accordance with various embodiments, the positive airway pressure device 100 provides positive airway pressure (even if the primary control parameter for the device 100 is therapeutic gas flow) at least during inhalation of the patient, the therapeutic gas flow and/or pressure to reduce sleep-disordered breathing such as snoring, hypopnea and/or apnea events. Control of the therapeutic gas flow and/or pressure delivered by the positive airway pressure device may take many forms. In some embodiments, the flow and/or pressure may be controlled by selectively controlling blower 40 speed. For example, FIG. 1 illustrates processor 30 coupled to a motor speed control circuit 52, which motor speed control circuit 52 is coupled to the motor 46. In alternative embodiments, the flow and/or pressure of therapeutic gas may be controlled by running the blower 40 at a relatively constant speed (i.e., no motor speed control circuit), and controlling the flow and/or pressure by control valve 55 (e.g., a butterfly valve) at the direction of the processor 30. While control valve 55 is illustrated on the outlet of the blower 40, the control valve 55 may be equivalently placed on the inlet of the blower 40. In yet other embodiments, a combination of controlling the blower 40 speed and the control valve 55 may be utilized.

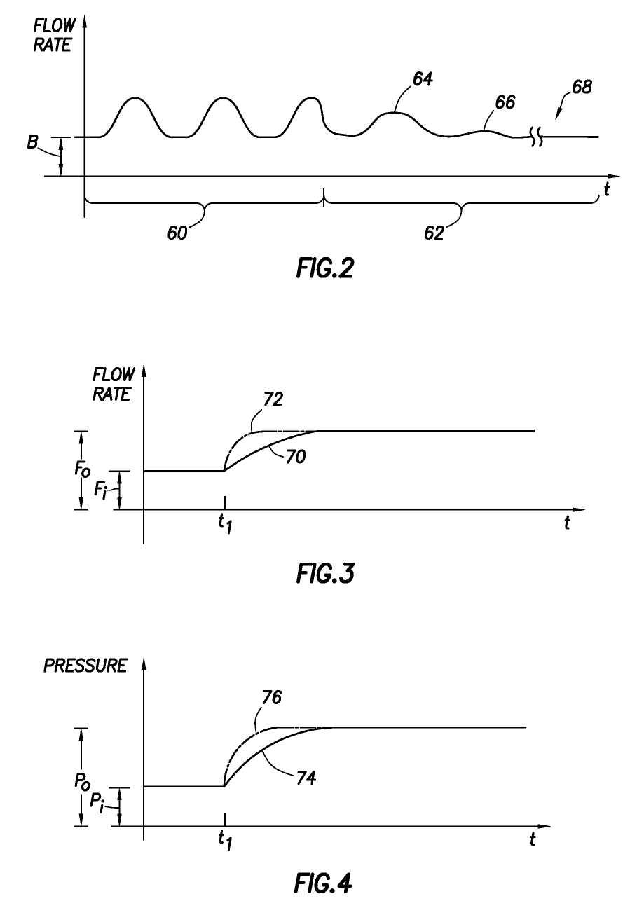

When the patient is sleeping and breathing normally, the therapeutic gas flow provided from the positive airway pressure device 100 to the patient is cyclical, as illustrated by the portion 60 in FIG. 2. The bias flow “B” in the figure is representative of the therapeutic gas that escapes through the vent 54 and any leaks at the interface between the mask 50 and the patient. The pressure applied during the period of time represented by FIG. 2 may be a constant (in continuous positive airway pressure (CPAP devices)), may lower during exhalation (bi-level devices), or may change breath-to-breath (in the so called “auto-titration” positive airway pressure devices, which may also come in continuous and bi-level varieties).

Portion 62 of FIG. 2 illustrates both a hypopnea and an apnea event. In particular, portion 62 illustrates hypopnea by waveforms 64 and 66, with hypopnea being abnormally slow or shallow breathing (as compared to the waveforms in portion 60). Portion 62 also illustrates an apnea event by portion 68, assuming the cessation of breathing represented by portion 68 lasts longer than ten seconds.

The difficulty faced by positive airway pressure devices, particularly the auto-titration positive airway pressure devices, is how to respond to an apnea event, as discussed in the Background. In accordance with some embodiments, the positive airway pressure device 100 is configured to gather data regarding whether the apnea event is central apnea or obstructive apnea, and in some embodiments make a determination. In embodiments where the positive airway pressure device makes a determination, the positive airway pressure device may also take action based on the determination. If the apnea is central apnea, the positive airway pressure device 100 lowers the peak applied pressure in the event the central apnea is over-pressure induced (but may also act as a ventilator during the apnea). If the apnea is obstructive, the positive airway pressure device raises applied pressure in an attempt to unblock the patient's airway. The discussion now turns to the data gathered and making the determination as to central or obstructive apnea.

Determining whether an apnea is central or obstructive, or at least gathering data such that a determination can be made, is based on the mechanics of an obstructive apnea and how the obstructive apnea affects airway volume perceived by the positive airway pressure device. In particular, an obstructive apnea is caused by a full or partial blockage of the upper airway, and in most cases the soft palate. When the upper airway is open, the volume of therapeutic gas accepted by the patient is the volume to fill the combination of the patient's lungs and upper airway (known as tidal volume). By contrast, when an obstruction is present, the volume of the lungs is substantially fluidly isolated from the positive airway pressure device, thus leaving only the volume of the upper airway upstream of the blockage. In accordance with some embodiments, gathering data indicative of whether the apnea is central apnea or obstructive apnea involves determining volume perceived by the positive airway pressure device. The mechanisms by which the determination is made may take many forms, depending on the abilities of the positive airway pressure device.

First consider a positive airway pressure device 100 such as illustrated in FIG. 1 having a flow sensor 42. When an apnea event is detected, the positive airway pressure device 100 first lowers applied pressure. The amount that the pressure is lowered may vary, but in some embodiments two inches of water below pressure applied during inhalation. In other embodiments, the applied pressure may be lowered to zero gauge pressure. Thereafter, the set point pressure is increased, and the volume of therapeutic gas accepted by the patient (taking into account the volume that escapes through the vent 54) is recorded. If the volume of therapeutic gas accepted by the patient's respiratory tract shows that no upper airway blockage is present (e.g., the volume accepted is more than one-third of the patient's tidal volume noted prior to the apnea event), then the apnea is central. In the case of a central apnea, the positive airway pressure device 100 may lower peak pressure, but may also continue to cyclically increase pressure (up to the new lowered maximum) and decrease pressure to act as a respirator during the central apnea event.

Conversely, if the volume accepted by the patient's respiratory tract shows an upper airway blockage (e.g., the volume accepted is less than one-third of the patient's tidal volume noted prior to the apnea event), then the apnea is obstructive. In the case of obstructive apnea, the positive airway pressure device 100 increases peak applied pressure in an attempt to open the airway.

Determining volume of the respiratory tract perceived by the positive airway pressure device 100 during the apnea event is merely illustrative. Any attribute indicative of volume may be sensed and used, even if the actual volume is not calculated. FIG. 3 shows an illustrative plot of therapeutic gas flow as a function of time during the period of time when the positive airway pressure device 100 has detected an apnea event and increased set point pressure (at time t1) as part of gathering data. In particular, during the apnea event, prior to increasing the set point pressure at time t1 the therapeutic gas flow value Fᵢ is the gas flow through the vent 54 and any leakage at the interface of the mask 50 and the patient. When set point pressure is increased (at time t1), therapeutic gas flow increases. The first curve 70 illustrates an exponential rise in therapeutic gas flow when the respiratory tract is open to airflow. Initially the therapeutic gas flow increases somewhat quickly, and as the lungs and upper airway fill with therapeutic gas at the increased pressure the therapeutic gas flow asymptotically approaches flow value F₀, being the flow through vent 54 at the increased pressure. The second curve 72 illustrates an exponential rise in therapeutic gas flow when the respiratory tract is blocked at the upper airway. Because of the blockage, the volume of additional therapeutic gas that can be accepted by the upper airway at the increased pressure is significantly less than the full respiratory tract. Thus, the therapeutic gas flow in the blocked case increases more quickly than the unblocked case, and then as the lungs and upper airway fill with therapeutic gas at the increased pressure the therapeutic gas flow asymptotically approaches flow value F₀, being the flow through vent 54 at the increased pressure.

In some embodiments, gathering data regarding or making a determination of whether an apnea event is central apnea or obstructive apnea may be based on how fast the therapeutic gas flow approaches the second (in this illustrative case higher) value. For example, the processor 30 may read a therapeutic gas flow rate just after the set point pressure is increased, may read a therapeutic gas flow rate again a predetermined amount of time later, and the difference in flow rate between these two points is indicative of volume. Alternatively, the processor 30 may observe the therapeutic gas flow rate, and determine how long it takes the therapeutic gas flow rate to reach the final value (with longer times indicative of no blockage, and shorter times indicative of blockage).

Each of curves 70 and 72 of FIG. 3 may be expressed in mathematical form, such as:

F(t)=Fᵢ+(Fₒ−Fᵢ)(1−e^{-λt}) (1)

Where F(t) is the therapeutic gas flow as a function of time t, Fᵢ is the therapeutic gas flow prior to increasing pressure set point, Fₒ is the final therapeutic gas flow, and λ (lambda) is a time constant. Thus, in some embodiments the processor 30 takes a series of data points and performs a curve fitting algorithm, such that the time constant is determined. Larger time constants are indicative of faster times to reach the final values and therefore smaller volumes (i.e., blockages). Smaller time constants are indicative of slower times to reach the final values and therefore larger volumes (i.e., absence of blockages in the upper airway). The tidal volume for the patient is also relevant, as the time constant for an unblocked airway of a small child may be approximately the same as the time constant for a blocked airway of a large adult.

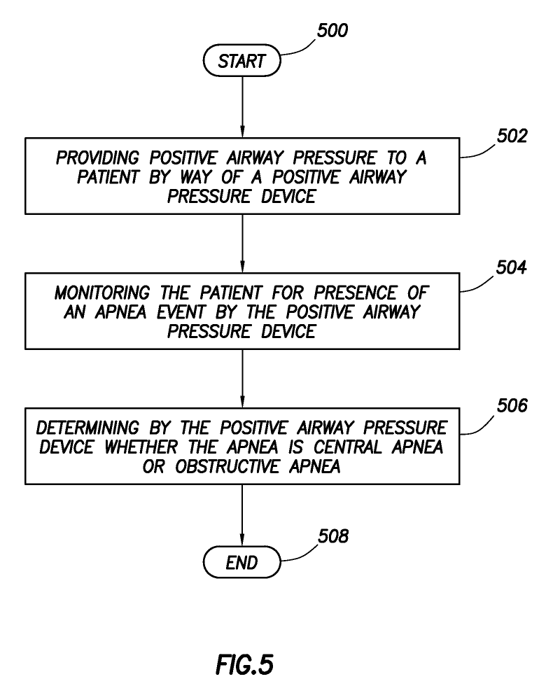

Now consider embodiments where, rather than using flow sensor 42, pressure sensor 44 is used. In embodiments using pressure sensor 44, the positive airway pressure device 100 monitors the pressure during the period of time when motor speed changes in response to changed set point pressure, the monitoring to gather data and/or to make a determination as to whether the apnea is central apnea or obstructive apnea. FIG. 4 shows an illustrative plot of applied pressure as a function of time during the period of time when the positive airway pressure device 100 has detected an apnea event and increased set point pressure (at time t1) as part of gathering data. In particular, during the apnea event, prior to increasing the set point pressure at time t1 the applied pressure value is P. When set point pressure is increased (at time t1), the applied pressure increases toward the new set point pressure. The first curve 74 illustrates an exponential rise in applied pressure when the respiratory tract is open to airflow. Initially the applied pressure increases somewhat quickly, and as the lungs and upper airway fill with therapeutic gas at the increased pressure the applied pressure asymptotically approaches pressure value P_{F}. The second curve 76 illustrates an exponential rise in applied pressure when the respiratory tract is blocked at the upper airway. Because of the blockage, the volume of additional therapeutic gas that can be accepted by the upper airway at the increased pressure is significantly less than the full respiratory tract, and final pressure P_{F} is reached more quickly.

In some embodiments, gathering data regarding or making a determination of whether an apnea event is central apnea or obstructive apnea may be based on how fast the applied pressure approaches the second (in this illustrative case higher) value. For example, the processor 30 may read applied pressure just after the set point pressure is increased, may read the applied pressure again a predetermined amount of time later, and the difference in applied pressure between these two points is indicative of volume. Alternatively, the processor 30 may observe the applied pressure, and determine how long it takes the applied pressure to reach the final value (with longer times indicative of no blockage, and shorter times indicative of blockage).

Each of curves 74 and 76 of FIG. 4 may be expressed in mathematical form, such as:

P(t)=Pᵢ+(Pₒ−Pᵢ)(1−e^{-λt}) (2)

Where P(t) is the applied pressure as a function of time t, Pᵢ is the applied pressure prior to increasing pressure set point, Pₒ is the final applied pressure, and λ (lambda) is a time constant. Thus, in some embodiments the processor 30 takes a series of data points and performs a curve fitting algorithm, such that the time constant is determined. Larger time constants are indicative of faster times to reach the final values and therefore smaller volumes (i.e., blockages). Smaller time constants are indicative of slower times to reach the final values and therefore larger volumes (i.e., absence of blockages in the upper airway). Here again, tidal volume for the patient is also relevant, as the time constant for an unblocked airway of a small child may be the same approximately the same as the time constant for a blocked airway of a large adult.

Now consider embodiments that utilize neither a flow sensor 42 nor a pressure sensor 44. Positive airway pressure devices 100 that do not use a flow or pressure sensor are usually the low-end devices; nevertheless, these devices too may be used to gather data, and in some cases make a determination, regarding whether an apnea is a central apnea or an obstructive apnea. In some embodiments, at least one of the electrical motor leads to the motor has associated therewith a current transformer 43 (FIG. 1). The illustrative current transformer 43 couples to the processor 30, and in particular to one of the A/D inputs 38. Current transformers give an indication of the net current flow in the wire or wires that pass through the current transformer. Electrical current draw by a motor coupled to a fan or blower is directly proportional to the therapeutic gas flow through the blower. In other words, the greater the electrical current drawn by the motor, the greater the therapeutic gas flow through the blower coupled to the motor, and vice versa.

Because current drawn by the motor is proportional to flow rate, when the device increases set point pressure, the electrical current as a function of time takes similar shape to the two curves of FIG. 3. If the patient's upper airway is unblocked, the rate at which the electrical current drawn by the motor approaches the second (in this case higher) value is relatively slow. If the patient's upper airway is blocked, the rate at which the electrical current drawing by the motor approaches the new (in this case higher) value is relatively fast. Thus, the same principles as discussed with respect to FIGS. 3 and 4 are equally applicable in the case of monitoring current drawn by the motor as the value indicative of volume.

In yet still other embodiments, the motor and blower may have an associated tachometer 43 (FIG. 1). The speed of the motor and blower are directly proportional to the flow rate through the blower. If the patient's upper airway is unblocked, when the device increases set point pressure, the rate at which the motor speed approaches the second (in this case higher) value is relatively slow. If the patient's upper airway is blocked, the rate at which the motor speed approaches the new (in this case higher) value is relatively fast. Thus, the same principles as discussed with respect to FIGS. 3 and 4 are equally applicable in the case of monitoring motor speed as the value indicative of volume.

The various embodiments discussed to this point have been based on determining a value indicative of the patient's respiratory volume based response to an increase in set point flow or pressure; however, in alternative embodiments the same determination is made based on decreases in pressure set point. By lowering set point pressure (e.g., turning off the motor and blower during the apnea event), a certain amount of gas flows out of the patient based on the volume of the upper airway and, if no blockage, the lungs. Some of the air escapes the through the vent in the patient mask, but some of the gas reverse flows through the positive airway pressure device. In these alternative embodiments, the value indicative of volume may be determined based on an attribute (e.g., pressure, volume, motor speed, motor current) of the reverse gas flow indicative of the volume.

FIG. 5 shows a method in accordance with at least some embodiments. In particular, the method starts (block 500) and proceeds to providing positive airway pressure to a patient by way of a positive airway pressure device (block 502). The positive airway pressure device monitors the patient for presence of an apnea event (block 504). If an apnea even is detected, the positive airway pressure device determines whether the apnea is central apnea or obstructive apnea (block 506), and the method ends (block 508). Determining whether the apnea is central apnea or obstructive apnea may take the many forms discussed above.

The various embodiments discussed to this point have been based on a positive airway pressure device 100 that treats the nostrils, and possibly the nose and mouth, as single breathing orifice; however, the various embodiments are not limited to positive airway pressure devices 100 with such an attribute. The inventor of the current specification is a co-inventor of U.S. Pat. No. 7,114,497 titled “Method and system of individually controlling airway pressure of a patient's nares.” In the '497 patent, therapeutic gas flow provided the nostrils (and in some cases the nostrils and mouth) are separately controlled; however, the various embodiments of the current specification are equally applicable to such a system.

The above discussion is meant to be illustrative of the principles and various embodiments of the present invention. Numerous variations and modifications will become apparent to those skilled in the art once the above disclosure is fully appreciated. For example, the various embodiments discussed to this point are in reference to changing set point pressure as part of gathering data, and making a determination, regarding whether an apnea is a central apnea or obstructive apnea; however, because of the controlled leak through vent 54, it is equivalent to change set point therapeutic gas flow as part of gathering data, and making a determination, regarding whether an apnea is a central apnea or obstructive apnea. It is intended that the following claims be interpreted to embrace all such variations and modifications.

What is claimed is:

1. A system comprising:
a processor; and
a blower mechanically coupled to a motor, the blower configured to fluidly couple to a breathing orifice of a patient, and the blower provides substantially all gas inhaled by the breathing orifice;
said processor configured to monitor for an apnea event of the patient, and when an apnea event is detected the processor is configured to gather data indicative of whether the apnea is central apnea or obstructive apnea.

2. The system of claim 1 wherein the processor is configured to determine whether the apnea is central apnea or obstructive apnea.

3. The system of claim 1 wherein to gather data the processor is configured to change a set point pressure of therapeutic gas provided to determine a value indicative of a volume of therapeutic gas supplied to the patient.

4. The system of claim 3 wherein to change pressure the processor is configured to increase set point pressure.

5. The system of claim 3 wherein to determine the value indicative of volume the processor is configured to determine a value indicative of a rate at which the pressure approaches second value.

6. The system of claim 5 wherein to determine the value indicative of the rate the processor is configured to determine a time constant of an exponential pressure response.

7. The system of claim 3 wherein to determine the value indicative of volume the processor is configured to determine a value indicative of a rate at which the flow to the patient approaches an asymptotic during the apnea event.

8. The system of claim 7 wherein to determine the value indicative of the rate the processor is configured to determine a time constant of an exponential flow response.

9. The system of claim 1 wherein to gather data the processor is configured to change a set point of pressure of therapeutic gas provided to the patient by the blower and determine a value indicative of electrical current drawn by the motor.

10. The system of claim 1 wherein to gather data the processor is configured to change a set point of pressure of therapeutic gas provided to the patient by the blower and determine a value indicative speed of the blower.

11. The system of claim 1 further comprising:
a pressure sensor fluidly coupled to the blower and electrically coupled to the processor;
when the processor monitors for an apnea event, the processor is configure to monitor, at least in part, therapeutic gas pressure sensed by the pressure sensor.

12. The system of claim 1 further comprising:
an flow sensor fluidly coupled to the blower and electrically coupled to the processor;
when the processor gathers data, the processor is configured to monitor, at least in part, therapeutic gas flow sensed by the flow sensor.

13. The system of claim 1 further comprising:
a current flow sensor electrically coupled to the processor, the current flow sensor senses electrical current flow of the motor of the blower;
when the processor gathers data, the processor is configured to monitor, at least in part, electrical current sensed by the current flow sensor.

14. The system of claim 1 further comprising:
a blower speed sensor coupled to the blower and electrically coupled to the processor;
when the processor gathers data, the processor is configured to monitor, at least in part, blower speed as sensed by the blower speed sensor.

15. A method comprising:
providing positive airway pressure to a patient by way of a positive airway pressure device, substantially all the gas inhaled by the patient is provided by the positive airway pressure device;
monitoring the patient for presence of an apnea event by the positive airway pressure device; and if an apnea event is detected
determining by the positive airway pressure device whether the apnea is central apnea or obstructive apnea.

16. The method of claim 15 wherein determining further comprises determining a value indicative of a volume of the respiratory tract as perceived by the positive airway pressure device.

17. The method of claim 15 wherein determining further comprises changing a flow of therapeutic gas applied to the patient during the apnea event and monitoring a rate of change of pressure applied by the positive airway pressure device.

18. The method of claim 17 wherein monitoring the rate of change of pressure further comprises determining a value indicative of a rate at which the pressure approaches an asymptotic value.

19. The method of claim 15 wherein determining further comprises changing a pressure of therapeutic gas applied to the patient during the apnea event and monitoring a rate of change of flow of therapeutic gas provided by the positive airway pressure device.

20. The method of claim 19 wherein monitoring the rate of change of flow further comprises determining a value indicative of a rate at which the flow approaches an asymptotic value.

21. The method of claim 15 wherein determining further comprises changing a set point of therapeutic gas supplied to the patient during the apnea event and monitoring a speed of a blower.

22. The method of claim 15 wherein determining further comprises changing a set point of therapeutic gas supplied to the patient during the apnea event and monitoring electrical current drawn by a motor coupled to a blower.

23. A computer-readable media storing a program that, when executed by a processor, causes the processor to:
control pressure of therapeutic gas applied to a patient in the treatment of sleep disordered breathing;
monitor the patient for presence of an apnea; and if an apnea is present
gather data indicative of whether the apnea is central apnea or obstructive apnea.

24. The computer-readable media of claim 23 wherein the program further causes the processor to determine whether the apnea is central or obstructive.

25. The computer-readable media of claim 23 wherein when the processor gathers data, the program further causes the processor to determine a value indicative of a volume of the respiratory tract.

26. The computer-readable media of claim 23 wherein when the processor gathers data, the program further causes the processor to change a flow of therapeutic gas applied to the patient during the apnea event and monitor a rate of change of pressure applied by the positive airway pressure device.

27. The computer-readable media of claim 23 wherein when the processor gathers data, the program further causes the processor to change a pressure of therapeutic gas applied to the patient during the apnea event and monitor a rate of change of flow of therapeutic gas provided by the positive airway pressure device.
